(19) United States
(12) Patent Application Publication
(10) Pub. No.: US 2011/0152872 A1
(43) Pub. Date: Jun. 23, 2011

(54) INTRAOCULAR LENS INJECTOR INCLUDING A SHAPED SPRING

(76) Inventors: William J. Seyboth, Rochester, NY (US); Jon P. Cullen, Churchville, NY (US); Graham W. Biddle, Ontario, NY (US); Emin Engin, Rochester, NY (US)

(21) Appl. No.: 12/645,577

(22) Filed: Dec. 23, 2009

(51) Int. Cl. A61F 9/007

(52) U.S. Cl. 606/107

(57) ABSTRACT

An intraocular lens (IOL) injector comprising an injector body having a lumen defined by a lumen wall, and a plunger adapted to slide within the lumen. The plunger is coupled to a spring having a plurality of coils, the plurality comprising at least one coil of a first outer diameter and at least one coil of second outer diameter that is different than the first outer diameter.

### FIELD OF INVENTION

The present invention relates to intraocular lens (IOL) injectors, and more particularly to IOL injectors providing plunger control using a shaped spring.

### BACKGROUND OF THE INVENTION

IOLs are artificial lenses used to replace or supplement the natural crystalline lenses of patients' when their natural lenses are diseased or otherwise impaired. IOLs may be placed in either the posterior chamber or the anterior chamber of an eye. IOLs come in a variety of configurations and materials.

Various instruments and methods for implanting such IOLs in an eye are known. Typically, an incision is made in a patient's cornea and an IOL is inserted into the eye through the incision. In one technique, a surgeon uses surgical forceps to grasp the IOL and insert it through the incision into the eye. While this technique is still practiced today, more and more surgeons are using IOL injectors, which offer advantages such as affording a surgeon more control when inserting an IOL into an eye and permitting insertion of IOLs through smaller incisions. Relatively small incision sizes (e.g., less than about 3 mm) are preferred over relatively large incisions (e.g., about 3.2 to 5+ mm) since smaller incisions have been attributed with reduced post-surgical healing time and reduced complications such as induced astigmatism.

In order for an IOL to fit through a small incision, it is typically folded and/or compressed by passing the IOL through a nozzle providing a funnel-shaped portion of the injector lumen and/or providing a compressor drawer. After the IOL exits an opening at the distal end of the nozzle and enters the eye, it assumes its original unfolded and/or uncompressed shape.

FIG. 1A is a schematic illustration of a conventional IOL injector 10. FIG. 1B is a cut away cross-section of a portion of the injector of FIG. 1A. Injector 10 comprises an injector body 20, a nozzle 60, and a plunger 30 that is substantially fully retracted within the injector body (i.e., the plunger is substantially fully withdrawn from the injector body). The plunger comprises a plunger body 32 and a plunger shaft 34. Typically, a plunger shaft is supported by an inner surface 36 of the lumen using one or more contact points (e.g., where the plunger rests due to gravity) resulting in undesirable instability and tilting (i.e., decentration) of the plunger within the lumen. Such instability and decentration may affect the interaction of the plunger tip and an IOL 50 during plunger actuation.

Many IOL insertion devices utilize a plunger spring 38 for plunger retraction after expression of the lens into the capsular bag. A feature 40 operates to limit travel of the spring in the longitudinal direction by interfering with the spring during actuation of the plunger. Accordingly, the spring is compressed against the feature during actuation of the plunger. In such embodiments, the spring may provide the one contact point with the lumen wall. FIG. 1C is an expanded, schematic illustration of a conventional spring as used in the injector of FIG. 1A.

The distance traveled by the plunger between full retraction and full extension (i.e., where the plunger is actuated far enough to fully deliver the IOL into an eye) is called the plunger stroke S.

A plunger may be particularly unstable at points of the stroke near full retraction, where interface I between the plunger and the injector body is relatively short. Plunger stability and centration with respect to the lumen may be particularly troublesome for molded plastic injectors, but also occurs in other injectors.

Stability and centration of the plunger 30 within the injector body is desirable to provide a user with a robust feeling when injecting an IOL, and to help ensure proper engagement of plunger tip 44 with IOL 50.

### SUMMARY

Aspects of the present invention are directed to improving centration and/or reducing tilting of the plunger. According to aspects of the invention, an injector is provided with a plunger spring having coils of different outer diameters to contact a lumen wall and thereby provide improved stability within an injector lumen.

An aspect of the invention is directed to an intraocular lens (IOL) injector comprising an injector body having a lumen defined by a lumen wall; and a plunger adapted to slide within the lumen, the plunger coupled to a spring having a plurality of coils, the plurality comprising at least one coil of a first outer diameter and at least one coil of a second outer diameter that is different than the first outer diameter.

In some embodiments, the plurality of coils comprising a first at least one coil having an inner diameter substantially equal to an outer diameter of the plunger, and the plurality of coils comprising a second at least one coil having an outer diameter substantially equal to an inner diameter of the lumen wall, the injector configured such that the coils contact the inner diameter of the lumen wall during actuation of the plunger. In some embodiments, the first at least one coil comprises at least two coils. In some embodiments, the second at least one coil comprises at least two coils.

The inner diameter of the lumen wall may be located in a non-lens-traversing portion of the injector body.

In some embodiments, the spring is sized and shaped such that a coil of the plurality of coils contacts the lumen wall at least two separated locations around a circumference of a portion of the lumen.

The spring may be sized and shaped to provide at least three-point contact with the lumen wall during actuation of the plunger.

In some embodiments, the coil of the plurality of coils and the lumen wall are configured to have substantially continuous contact around a circumference of the lumen wall at a portion of the lumen along the longitudinal axis.

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1A is a schematic illustration of a conventional IOL injector;

FIG. 1B is a cut away cross section of a portion of the injector of FIG. 1A;

FIG. 1C is a schematic illustration of a conventional spring as used in the injector of FIG. 1A;

FIG. 2 is a cutaway, schematic illustration of an example of an IOL injector according to aspects of the present invention;

FIG. 3 is a schematic illustration of the plunger and spring of the IOL injector of FIG. 2;

FIG. 4 is a schematic illustration of the spring of the IOL injector of FIG. 2; and

FIG. 5 is a cross sectional view of another embodiment of an IOL injector, showing a portion of the lumen at a selected longitudinal location along the injector's length.

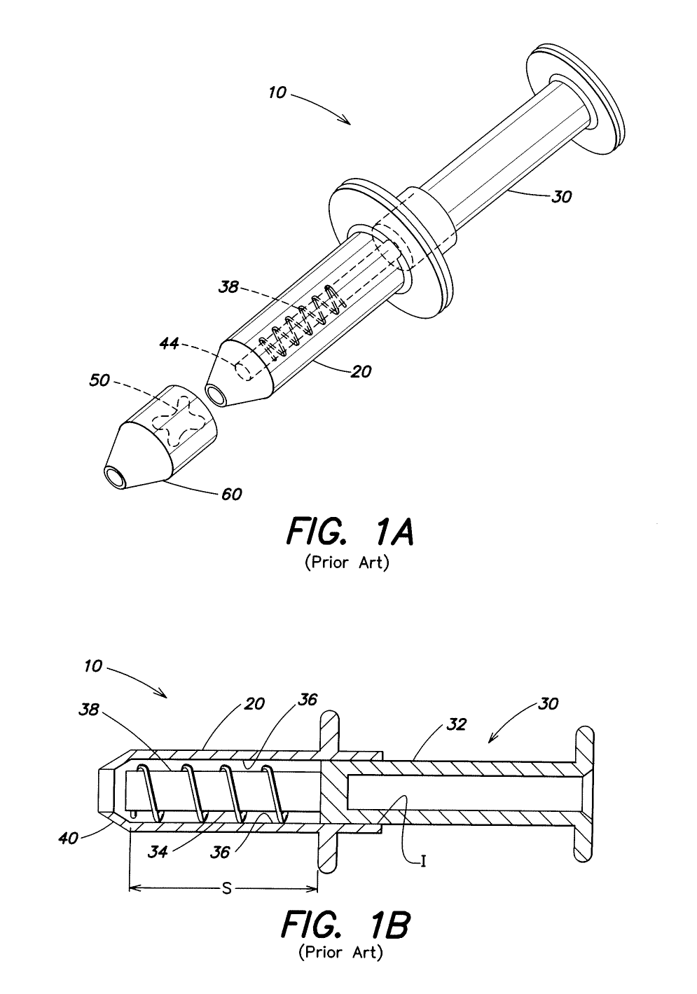

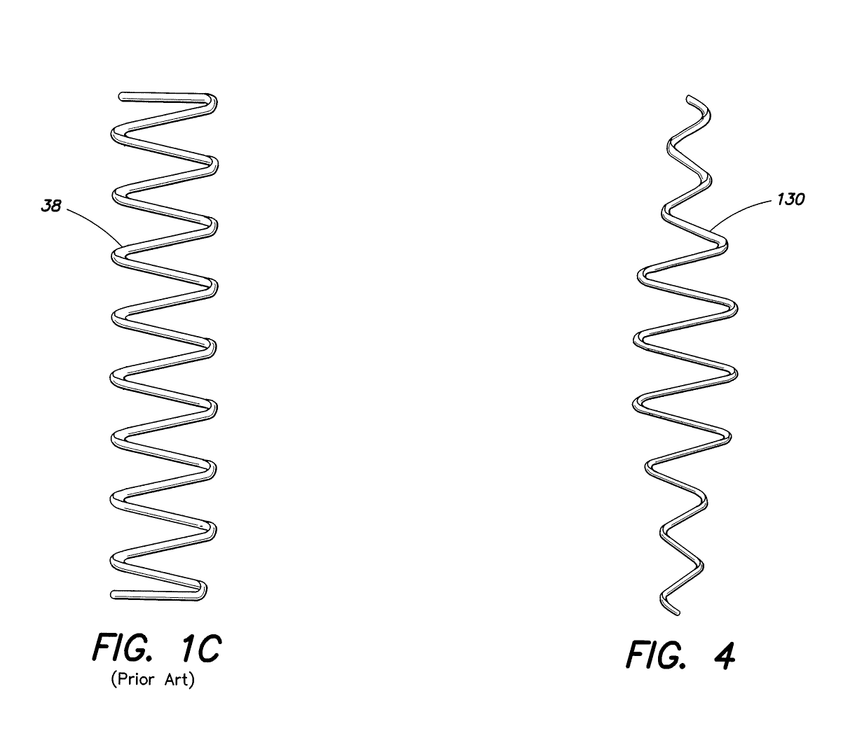

### DETAILED DESCRIPTION

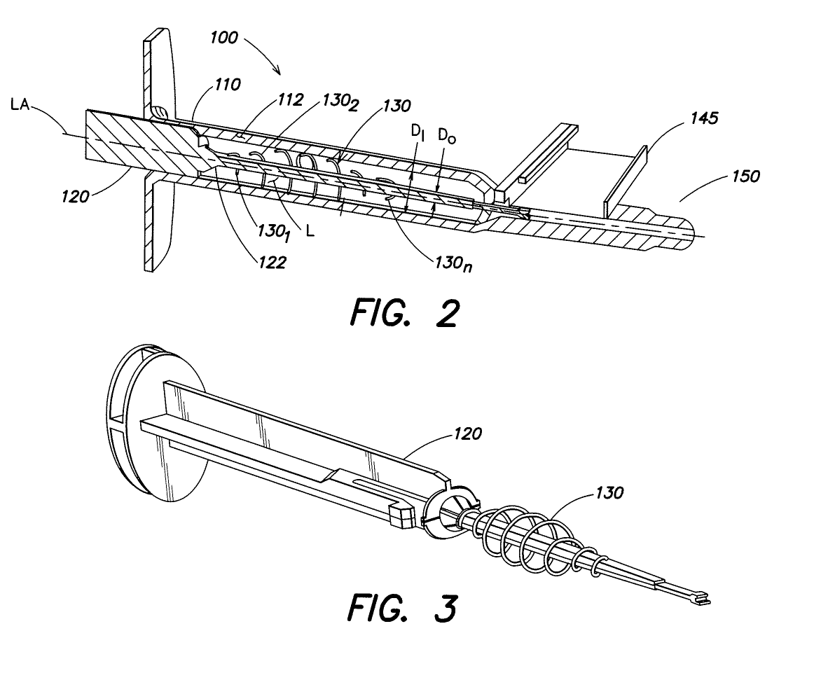

FIG. 2 is a cutaway, schematic illustration of an example of an IOL injector 100 according to aspects of the present invention. Injector 100 comprises an injector body 110 and a plunger 120 having a plunger spring 130 coupled thereto. It will be appreciated that an injector according to aspects of the present invention may be configured substantially similarly to any suitable prior art injector except the plunger spring is suitably shaped.

The injector body has a lumen L defined by a lumen wall 112. The lumen may have any suitable cross-sectional shape which may change along the longitudinal axis of the injector. For example, a lumen may have a round, oval, triangular, quadrilateral or elliptical shape or other suitable shape, and may change shapes along the longitudinal axis LA.

Plunger 120 is adapted to slide within lumen L. The plunger is coupled to spring 130. The plunger has a plurality of coils 130₁-130ₙ of different outer diameters (i.e., at least two coils of the plurality of coils have different diameters than one another). The presence of different diameters allows a selected one or more of the coils to maintain contact with the plunger and another selected one or more of the coils to maintain contact with the lumen wall thereby providing stability of the plunger within the lumen. FIG. 3 is a schematic illustration of the plunger and spring 130 of IOL injector 100 in FIG. 2. FIG. 4 is a schematic illustration of spring 130 of the IOL injector in FIG. 2.

It will be appreciated that, in some embodiments, at least one of the plurality of coils 130₁-130ₙ has an inner diameter substantially equal to an outer diameter D_{O} of the plunger. At least one of the plurality of coils has an outer diameter substantially equal to an inner diameter D_{I} of the lumen wall 112. The injector is configured such that the extremes of the outer diameter of the coils contact the inner diameter of the lumen wall during actuation of the plunger. It will be appreciated that although, in some embodiments, an outer diameter of at least one of the plurality of coils is substantially equal to an inner diameter D_{I} of the lumen wall 112, said outer diameter may be equal to or greater than inner diameter D_{I} of the lumen wall 112; it will be further appreciated that by appropriately selecting the size of said outer diameter of the at least one of the plurality of coils relative to the inner diameter of the lumen wall, it is possible to select a resistance to axial movement of the plunger moving through the lumen, thereby imparting a feel to a person depressing the plunger. The term “diameter” is used in this document to mean a radial dimension and is not limited to shapes having a circular cross-section.

Although in the embodiment illustrated in FIG. 2 the spring and lumen wall have the same shape (e.g., a circle of a particular radius) and therefore they make contact substantially continuously around a circumference of the lumen wall at a portion of the lumen along the longitudinal axis, in some embodiments, the spring and lumen wall do not have the same shape. It will be appreciated that the term “around a circumference of the lumen wall at a portion of the lumen” is used because the spring is helical and does not have form a closed cross-sectional shape at a single longitudinal location (i.e., a coil of a spring has a length).

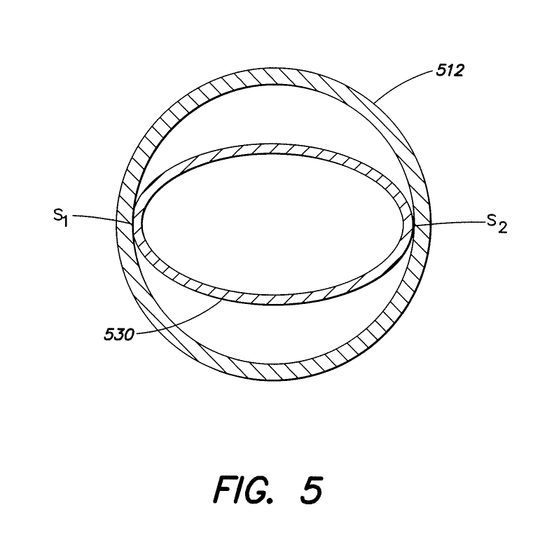

FIG. 5 is a cross sectional view of another embodiment of an IOL injector in which a spring 530 and lumen wall 512 do not have the same shape. FIG. 5 shows a portion of the lumen at a selected longitudinal location along the injector's length. As shown in FIG. 5, a spring coil 530 is sized and shaped to contact the lumen wall at least two separated locations S₁ and S₂ around the circumference of the portion of the lumen. (i.e., there is a location between S₁ and S₂ where the spring does not make contact with the lumen wall). For example, the spring may have an oval shape with only the major axis having a diameter equal to the inner diameter of a circularly cross-sectioned lumen wall. Alternatively, the spring may have a circular shape with a diameter equal to the diameter of the minor axis of a lumen having an ovoid shape.

Referring again to FIG. 2, in the illustrated embodiment, the injector is configured such that the location where the lumen wall and the spring make contact is located in a portion of the injector body where the IOL will not traverse. For example, in FIG. 2, the IOL is loaded at loading bay 145 where the IOL is compressed, for example, by a drawer prior to actuation of the plunger and ejection of the IOL into an eye through tip 150. Further details of an injector having such a drawer are given in U.S. Pat. No. 6,491,697. The substance of said patent is hereby incorporated by reference in its entirety. In other embodiments, the injector is configured such that the location where the lumen wall and the spring make contact is located in a portion of the injector body where the IOL will traverse (not shown).

In some embodiments, the spring is sized and shaped to provide at least three-point contact with a portion of the lumen wall during actuation of the plunger. For example, the lumen wall may have a triangular cross-sectional shape and the spring is circular.

The proximal end of the spring may be connected to the plunger at location 122 or may be arranged to press against a surface of the plunger at location 122 (during actuation) without attachment to the plunger.

The spring may be made of any suitable material. The material should be able to flex and return to a non-flexed state without fracture. For example, the spring may be made of stainless steel, copper, plastic or ceramic. The spring may have any suitable total number of coils and any suitable number of coils of any given inner or outer diameter. The spring may be a barrel spring. For example, the plunger spring may have two coils (proximal coils) having an inner diameter equal to an outer diameter of the plunger at the proximal end of the spring, two coils (distal coils) having an inner diameter equal to an outer diameter of the plunger at the distal end of the spring, and four coils intermediate the proximal coils and distal coils which have an outer diameter equal to (or greater than) an inner diameter of the lumen wall. In some embodiments, the proximal or the distal coils that have an inner diameter equal to the outer diameter of the plunger may be omitted, such that the coils that have an outer diameter equal to (or greater than) an inner diameter of the lumen wall are located at the distal end of the spring or the proximal end of the spring.

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. An intraocular lens (IOL) injector comprising:
an injector body having a lumen defined by a lumen wall; and
a plunger adapted to slide within the lumen, the plunger coupled to a spring having a plurality of coils, the plurality comprising at least one coil of a first outer diameter and at least one coil of a second outer diameter that is different than the first outer diameter.

2. The injector of claim 1, wherein the plurality of coils comprises a first at least one coil having an inner diameter substantially equal to an outer diameter of the plunger, and the plurality of coils comprises a second at least one coil having an outer diameter substantially equal to an inner diameter of the lumen wall, the injector configured such that the second coil contacts the inner diameter of the lumen wall during actuation of the plunger.

3. The injector of claim 2, wherein the inner diameter of the lumen wall is located in a non-lens-traversing portion of the injector body.

4. The injector of claim 1, wherein the spring is sized and shaped such that a coil of the plurality of coils contacts the lumen wall at least two separated locations around a circumference of a portion of the lumen.

5. The injector of claim 1, wherein the spring is sized and shaped such that a coil of the plurality of coils contacts the lumen wall at least three separated locations around a circumference of a portion of the lumen.

6. The injector of claim 1, wherein a coil of the plurality of coils and the lumen wall are configured to substantially continuous contact around a circumference of the lumen wall at a portion of the lumen along the longitudinal axis.

7. The injector of claim 1, wherein the plurality of coils comprises an at least one coil having an outer diameter equal to or greater than an inner diameter of the lumen wall, the injector configured such that the at least one coil contacts the inner diameter of the lumen wall during actuation of the plunger.
